(19) United States
(12) Patent Application Publication
(10) Pub. No.: US 2014/0288316 A1
(43) Pub. Date: Sep. 25, 2014

(54) METHOD FOR PREPARING 2,6-DIFLUOROACETOPHENONES

(71) Applicant: E I DU PONT DE NEMOURS AND COMPANY, Wilmington, DE (US)

(72) Inventors: Ty Wagerle, West Chester, PA (US); John P. Daub, Wilmington, DE (US)

(21) Appl. No.: 14/357,616

(22) PCT Filed: Nov. 15, 2012
(86) PCT No.: PCT/US2012/065158

§ 371 Date: May 12, 2014

Related U.S. Application Data
(60) Provisional application No. 61/566,861, filed on Dec. 5, 2011.

(51) Int. Cl. C07C 49/807

(52) U.S. Cl. CPC C07C 49/807
USPC 549/267; 568/315; 568/442

(57) ABSTRACT

Disclosed are methods for preparing compounds of Formula 1 utilizing an intermediate of Formula 4 or an intermediate of Formula 6.

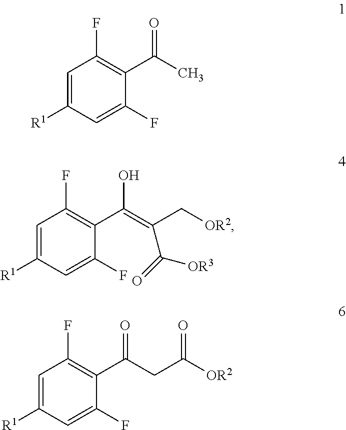

Also disclosed are compounds of Formula 4.

### FIELD OF THE INVENTION

This invention pertains to methods for preparing certain 2,6-difluoroacetophenones. The present invention also relates to intermediates for the aforedescribed methods.

### BACKGROUND OF THE INVENTION

Preparation of certain 2,6-difluoroacetophenones are known in the chemical literature. However, the need continues for new or improved methods suitable for rapidly and economically providing 2,6-difluoroacetophenones,

### SUMMARY OF THE INVENTION

The present invention provides a method for preparing a compound of Formula 1

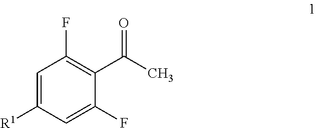

wherein
R¹ is H, F, Cl or Br;
   comprising (A) contacting a compound of Formula 2

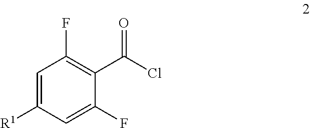

with a compound of Formula 3

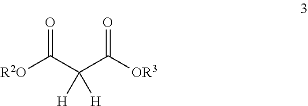

wherein
R² and R³ are independently CH₃, CH₂CH₃, CH₂CH═CH₂ or R² and R³ groups can be taken together as —C(CH₃)₂— to form a ring
   and an alkaline earth salt of a strong acid in the presence of a tertiary amine base and an aprotic solvent to form a salt of a compound of Formula 4

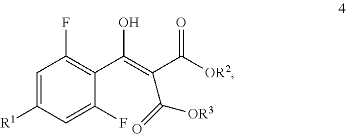

(B) contacting the salt of the compound of Formula 4 with water and an acid to form the compound of Formula 4 or tautomer thereof, and (C) contacting the compound of Formula 4 with water and heating to a temperature in the range of 85 to 80° C. to give the compound of Formula 1.

The present invention also relates to novel compounds of Formula 4

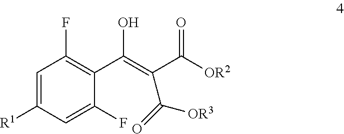

wherein
R¹ is H, F, Cl or Br; and
R² and R³ are independently CH₃, CH₂CH₃, CH₂CH═CH₂ or R² and R³ groups can be taken together as —C(CH₃)₂— to form a ring.

The present invention also provides a method for preparing a compound of Formula 1

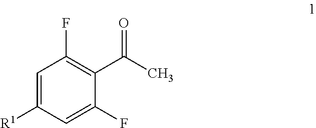

wherein
R¹ is H, F, Cl or Br;
   comprising (A) contacting a compound of Formula 2

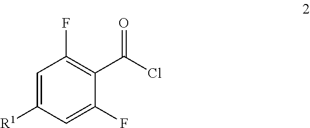

with a compound of Formula 5

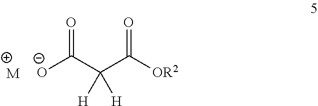

wherein
R² is CH₃, CH₂CH₃ or CH₂CH═CH₂ and
M is Li, Na or K
   and an alkaline earth salt of a strong acid in the presence of a tertiary amine base and an aprotic solvent to form a salt of a compound of Formula 6

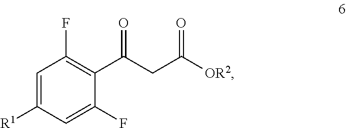

(B) contacting the salt of the compound of Formula 6 with an acid and water to form the compound of Formula 6 or tautomer thereof,
and (C) contacting the compound of Formula 6 with water and heating to a temperature in the range of 85 to 180° C. to give the compound of Formula 1.

### DETAILS OF THE INVENTION

As used herein, the terms “comprises,” “comprising,” “includes,” “including,” “has,” “having,” “contains” or “containing,” or any other variation thereof are intended to cover a non-exclusive inclusion. For example, a composition, a mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present). A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

The term “ambient temperature” or “room temperature” as used in this disclosure refers to a temperature between about 18° C. and about 28° C.

One skilled in the art recognizes that compounds of Formula 4 can exist in equilibrium with one or more of its respective tautomeric counterparts. Unless otherwise indicated, reference to a compound by one tautomer description structure or name) is to be considered to include all tautomers. For example, in Formula 4 when R² and R³ are different, then reference to the tautomeric form depicted by Formula 4¹ also includes the tautomeric forms depicted by Formula 4² through Formula 4⁷.

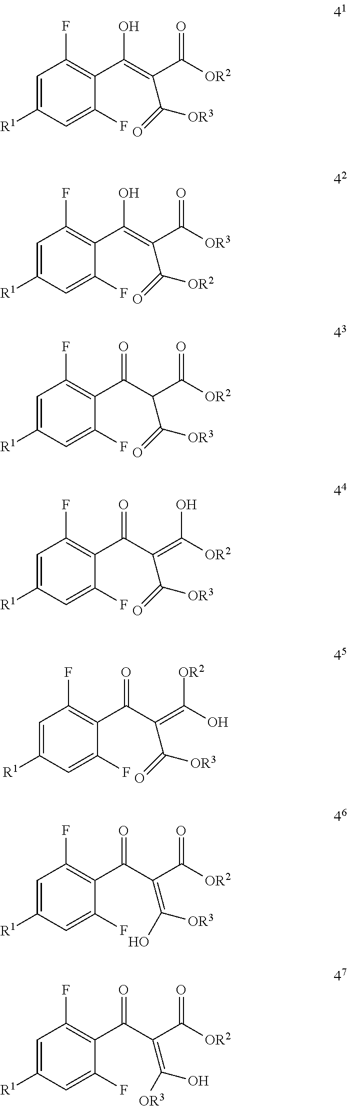

One skilled in the art recognizes that compounds of Formula 6 can exist in equilibrium with one or more of its respective tautomeric counterparts. Unless otherwise indicated, reference to a compound by one tautomer description (structure or name) is to be considered to include all tautomers. For example, in Formula 6 when R² and R³ are different, then reference to the tautomeric form depicted by Formula 6¹ also includes the tautomeric forms depicted by Formula 6² through Formula 6⁵.

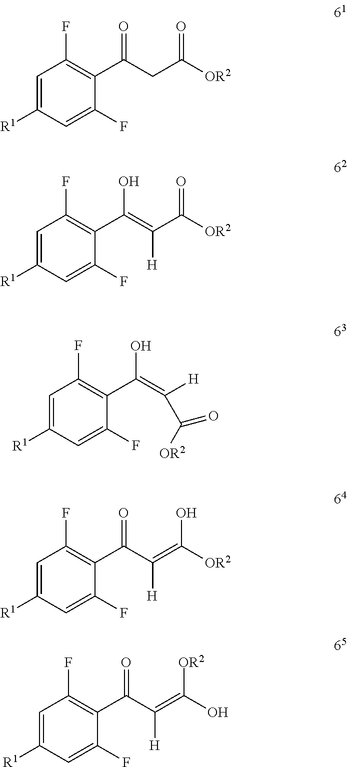

A compound of Formula 3 wherein R² and R³ are ethyl is diethyl malonate or 1,3-diethyl propanedioate. A compound of Formula 5 wherein R² is ethyl and NI is potassium is ethyl malonate, potassium salt or potassium 1-ethyl propanedioate. A compound of Formula 4 wherein R¹ is H; and R² and R³ are ethyl is 1,3-diethyl 2-(2.6-difiuorobenzoyl)propanedioate (keto form Formula 4³) or 1,3-diethyl 2-[(2,6-difluorophenyl)hydroxymethylene]propanedioate (enol form Formula 4¹). A compound of Formula 1 wherein R¹ is H is 2,6-difluoroacetophenone or 1-(2,6-difluorophenyl)ethanone.

Embodiments of the present invention include:

### Embodiment A1

The method described in the Summary of the Invention for preparing the compound of Formula 1 comprising, (A) contacting a compound of Formula with a compound of Formula 3 and an alkaline earth salt of a strong acid in the presence of a tertiary amine base and an aprotic solvent to form a salt of a compound of Formula 4, (B) contacting the salt of the compound of Formula 4 with an acid and water to form the compound of Formula 4 or tautomer thereof, and (C) contacting the compound of Formula 4 with water and heating to a temperature in the range of 85 to 180° C. to give the compound of Formula 1.

### Embodiment A2

The method of Embodiment A1 wherein R¹ is H, F or Cl.

### Embodiment A3

The method of Embodiment A2 wherein R¹ is H.

### Embodiment A4

The method of any one of Embodiments A1 through A3 wherein R² and R³ are independently CH₃ or CH₂CH₃.

### Embodiment A5

The method of Embodiment A4 wherein R² and R³ are CH₂CH₃.

### Embodiment A6

The method of any one of Embodiments A1 through A5 wherein the alkaline earth salt of a strong acid is magnesium chloride or calcium chloride.

### Embodiment A7

The method of Embodiment A6 wherein the alkaline earth salt of a strong acid is magnesium chloride.

### Embodiment A8

The method of any one of Embodiments A1 through A7 wherein the tertiary amine base is selected from the group consisting of tributylamine, triethylamine, diisopropylethylamine, pyridine, picolines, lutidines, N,N-dimethylaniline and N,N-diethylaniline.

### Embodiment A9

The method of Embodiment A8 wherein the tertiary amine base is tributylamine, triethylamine, pyridine, 2-picoline, 2,6-lutidine or N,N-diethylaniline.

### Embodiment A10

The method of Embodiment A9 wherein the tertiary amine base is triethylamine.

### Embodiment A11

The method of any one of Embodiments A1 through A10 wherein the aprotic solvent is chlorobenzene, toluene, xylenes, dichloromethane, tetrahydrofuran, acetonitrile or ethyl acetate.

### Embodiment A12

The method of Embodiment A11 wherein the aprotic solvent is chlorobenzene or ethyl acetate.

### Embodiment A13

The method of Embodiment A12 wherein the aprotic solvent is chlorobenzene.

### Embodiment A14

The method of any one of Embodiments A1 through A13 wherein in step (A) the compound of Formula 3 and the alkaline earth salt of a strong acid in the presence of the aprotic solvent are contacted first with the tertiary amine base and allowed to form a reaction mixture (enolate) and then the reaction mixture (enolate) is contacted with the compound of Formula 2 to thrill the salt of the compound of Formula

### Embodiment A15

The method of Embodiment A14 wherein in step (A) the temperature is in the range of 0 to 25° C.,

### Embodiment A16

The method of Embodiment A15 wherein in step (A) the temperature is in the range of 20 to 25° C.

### Embodiment A17

The method of any one of Embodiments A1 through A16 wherein the molar ratio of the compound of Formula 3 to the compound of Formula 2 is in the range of 1.5:1.0 to 1.0:1.0.

### Embodiment A18

The method of any one of Embodiments A1 through A17 wherein the molar ratio of the alkaline earth salt of a strong acid to the compound of Formula 2 is in the range of 3.5:1.0 to 3.0:1.0.

### Embodiment A19

The method of any one of Embodiments A1 through A18 wherein the molar ratio of the tertiary amine base to the compound of Formula 2 is in the range of 3.5:1.0 to 3.0:1.0.

### Embodiment A20

The method of any one of Embodiments A1 through A19 wherein in step (B) the salt of the compound of Formula 4 is contacted with water and the acid to thrill the compound of Formula 4 or tautomer thereof

### Embodiment A21

The method of any one of Embodiments A1 through A20 wherein the acid is hydrochloric acid,

### Embodiment A22

The method of Embodiments A20 and A21 wherein in step (B) the temperature is in the range of 0 to 25° C.

### Embodiment A23

The method of Embodiment A22 wherein in step (B) the temperature is in the range of 0 to 15° C.

### Embodiment A24

The method of any one of Embodiments A20 through A23 wherein in step (B) the molar ratio of the acid to the compound of Formula 2 is in the range of 3.0:1.0 to 4.0:1.0.

### Embodiment A25

The method of any one of Embodiments A1 through A24 wherein in step (C) the compound of Formula 4 is contacted with water and heated to a temperature in the range of 85 to 180° C. to give the compound of Formula 1.

### Embodiment A26

The method of Embodiment A25 wherein in step (C) the compound of Formula 4 is contacted with at least 2 equivalents of water for every equivalent of the compound of Formula 2.

### Embodiment A27

The method of Embodiments A25 and A26 wherein in step (C) the compound of Formula 4 is contacted with water in a pressure reactor,

### Embodiment A28

The method of any one of Embodiments A25 through A27 wherein in step (C) the temperature is in the range of 130 to 160° C.

### Embodiment A29

The method of Embodiment A28 wherein in step (C) the temperature is in the range of 135 to 155° C.

### Embodiment A30

The method of any one of Embodiments A1 through A24 wherein in step (C) the compound of Formula 4 is contacted with water in the presence of an acid and heated to a temperature in the range of 85 to 130° C. to give the compound of Formula 1.

### Embodiment A31

The method of Embodiment A30 wherein in step (C) the compound of Formula 4 is contacted with at least 10 mole of the acid and at least 2 equivalents of water for every equivalent of the compound of Formula 2.

### Embodiment A32

The method of Embodiments A30 and A31 wherein in step (C) the acid is sulfuric acid, arylsulfonic acids, carboxylic acids or mixtures thereof.

### Embodiment A33

The method of any one of Embodiments A30 through A32 wherein in step (C) the acid is sulfuric acid, acetic acid or mixtures thereof

### Embodiment B1

The method described in the Summary of the Invention for preparing the compound of Formula 1 comprising, (A) contacting a compound of Formula 2 with a compound of Formula 5 and an alkaline earth salt of a strong acid in the presence of a tertiary amine base and an aprotic solvent to form a salt of a compound of Formula 6, (B) contacting the salt of the compound of Formula 6 with an acid and water to form the compound of Formula 6 or tautomer thereof, and (C) contacting the compound of Formula 6 with water and heating to a temperature in the range of 85 to 180° C. to give the compound of Formula 1.

### Embodiment B2

The method of Embodiment B1 wherein R¹ is H, F or Cl.

### Embodiment B3

The method of Embodiment B2 wherein R¹ is H,

### Embodiment B4

The method of any one of Embodiments B1 through B3 wherein R² is CH₃ or CH₂CH₃.

### Embodiment B5

The method of Embodiment B4 wherein R² is CH₂CH₃.

### Embodiment B6

The method of any one of Embodiments B1 through B5 wherein M is Na or K.

### Embodiment B7

The method of Embodiment B6 wherein M is K.

### Embodiment B8

The method of any one of Embodiments B1 through B7 wherein the alkaline earth salt of a strong acid is magnesium chloride or calcium chloride.

### Embodiment B9

The method of Embodiment B8 wherein the alkaline earth salt of a strong acid is magnesium chloride.

### Embodiment B10

The method of any one of Embodiments B1 through B9 wherein the tertiary amine base is selected from the group consisting of tributylamine, triethylamine, diisopropylethylamine, pyridine, picolines, lutidines, N,N-dimethylaniline and N,N-diethylaniline.

### Embodiment B11

The method of Embodiment B10 wherein the tertiary amine base is tributylamine, triethylamine, pyridine, 2-picoline, 2,6-lutidine or N,N-diethylaniline.

### Embodiment B12

The method of Embodiment B11 wherein the tertiary amine base is triethylamine.

### Embodiment B13

The method of any one of Embodiments B1 through B12 wherein the aprotic solvent is chlorobenzene, toluene, xylenes, dichloromethane, tetrahydrofuran, acetonitrile or ethyl acetate.

### Embodiment B14

The method of Embodiment B13 wherein the aprotic solvent is chlorobenzene or ethyl acetate.

### Embodiment B15

The method of Embodiment B14 wherein the aprotic solvent is ethyl acetate.

### Embodiment B16

The method of any one of Embodiments B1 through B15 wherein in step (A) the compound of Formula 5 and the alkaline earth salt of a strong acid in the presence of the aprotic solvent are contacted first with the tertiary amine base and allowed to form a reaction mixture (enolate and then the reaction mixture (enolate) is contacted with the compound of Formula 2 to form the salt of the compound of Formula 6.

### Embodiment B17

The method of Embodiment B16 wherein in step (A) the temperature is in the range of 0 to 50° C.

### Embodiment B18

The method of Embodiment B17 wherein in step (A) the temperature is in the range of 20 to 50° C.

### Embodiment B19

The method of any one of Embodiments B1 through B18 wherein the molar ratio of the compound of Formula 5 to the compound of Formula 2 is in the range of 1.5:1.0 to 1.0:1.0.

### Embodiment B20

The method of any one of Embodiments B1 through B19 wherein the molar ratio of the alkaline earth salt of a strong acid to the compound of Formula 2 is in the range of 3.5:1.0 to 3.0:1.0.

### Embodiment B21

The method of any one of Embodiments B1 through B20 wherein the molar ratio of the tertiary amine base to the compound of Formula 2 is in the range of 3.5:1.0 to 3.0:1.0.

### Embodiment B22

The method of any one of Embodiments B1 through B21 wherein in step (B) the salt of the compound of Formula 6 is contacted with water and the acid to form the compound of Formula 6 or tautomer thereof.

### Embodiment B23

The method of any one of Embodiments B1 through B22 wherein the acid is hydrochloric acid.

### Embodiment B24

The method of Embodiments B22 and B23 wherein in step (B) the temperature is in the range of 0 to 25° C.,

### Embodiment B25

The method of Embodiment B24 wherein in step (B) the temperature is in the range of 0 to 15° C.

### Embodiment B26

The method of any one of Embodiments B22 through B25 wherein in step (B) the molar ratio of the acid to the compound of Formula 2 is in the range of 3.0:1.0 to 4.0:10.

### Embodiment B27

The method of any one of Embodiments B1 through B26 wherein in step (C) the compound of Formula 6 is contacted with water and heated to a temperature in the range of 85 to 180° C. to give the compound of Formula 1.

### Embodiment B28

The method of Embodiment B27 wherein in step (C) the compound of Formula 6 is contacted with at least one equivalent of water for every equivalent of the compound of Formula 2.

### Embodiment B29

The method of Embodiments B27 and B28 wherein in step (C) the compound of Formula 6 is contacted with water in a pressure reactor.

### Embodiment B30

The method of any one of Embodiments B27 through B29 wherein in step (C) the temperature is in the range of 130 to 160° C.

### Embodiment B31

The method of any one of Embodiments B1 through B26 wherein in step (C) the compound of Formula 6 is contacted with water in the presence of an acid and heated to a temperature in the range of 85 to 130° C. to give the compound of Formula 1.

### Embodiment B32

The method of Embodiment B31 wherein in step (C) the compound of Formula 6 is contacted with at least 10 mole % of the acid and at least 2 equivalents of water for every equivalent of the compound of Formula 2.

### Embodiment B33

The method of Embodiments B31 and B32 wherein in step (C) the acid is sulfuric acid, arylsulfonic acids, carboxylic acids or mixtures thereof

### Embodiment B34

The method of any one of Embodiments B31 through B33 wherein in step (C) the acid is sulfuric acid, acetic acid or mixtures thereof.

### Embodiment C1

A compound of Formula 4 wherein R¹ is H, F, Cl or Br; and R² and R³ are independently CH₃, CH₂CH₃, CH₂CH═CH₂ or R² and R³ groups can be taken together as —C(CH₃)₂— to form a ring.

### Embodiment C2

A compound of Formula 4 wherein R¹ is H, F or Cl; and R² and R³ are independently CH₃ or CH₂CH₃.

### Embodiment C3

A compound of Formula 4 wherein R¹ is H; and R² and R³ are CH₂CH₃ [also named 1,3-diethyl 2-(2,6-difluorobenzoyl)propanedioate (in keto form) or 1,3-diethyl 2-[(2,6-difluorophenyl)hydroxymethylene]propanedioate (in enol form].

### Embodiment C4

A compound of Formula 4 useful for preparing a compound of Formula 1 in the method described in the Summary of the Invention and Embodiment A1.

Embodiments of this invention, including Embodiments A1-A33, B1-B34 and C1-C4 above as well as any other embodiments described herein, can be combined in any manner, and the descriptions of variables in the embodiments pertain not only to the aforedescribed methods for preparing compounds of Formulae 1, but also to the starting compounds and intermediate compounds useful for preparing the compounds of Formulae 1 by these methods.

In the following Schemes 1-6 the definition of R¹, R², R³ and M in the compounds of Formulae 1 through 6 are as defined above in the Summary of the Invention and description of Embodiments unless otherwise indicated.

In the method of the invention, a compound of Formula 3 and a compound of Formula 2 are reacted to form a diester intermediate of Formula 4. The diester intermediate of Formula 4 is hydrolyzed and decarboxylated to give the compound of Formula 1. This sequence is shown in Schemes 1, 2 and 3.

Step C of the method of the invention involves hydrolysis of the ester groups in an intermediate of Formula 4 and decarboxylation of the resultant carboxylic acid functional groups to give a compound of Formula 1 as shown in Scheme 1.

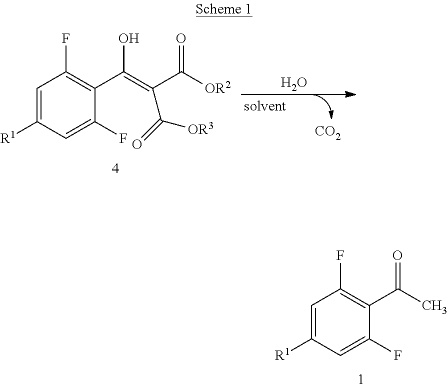

The hydrolysis of the ester groups in the compound of Formula 4 can be accomplished under neutral conditions with water. The hydrolysis reaction can be run under a broad range of temperatures. Temperatures in the range of 85 to 180° C. are particularly useful. The lower the temperature used for the hydrolysis, the longer the reaction will take to complete. Therefore temperatures in the range of 130 to 160° C. are especially useful in order to complete the hydrolysis in a reasonable period of time (less than an hour to several hours). The reaction is conducted in Examples 1 and 4 between 135 to 155° C. and it is complete in 1 to 2 hours. When the ester hydrolysis/decarboxylation is conducted with water under neutral conditions at temperatures above the boiling point of water, it is especially useful to run the reaction in a pressure reactor. The pressure reactor can be equipped with a back pressure regulator to enable maintenance of constant pressure while carbon dioxide is evolved and a condenser to return water or solvent to the reaction mixture containing the intermediate of Formula 4,

The hydrolysis reaction requires at least two equivalents of water for every equivalent of a compound of Formula 4, however, an excess of water can be useful to decrease reaction time. The hydrolysis/decarboxylation reaction can be conducted either in a one phase homogeneous solution or a two phase system. The solvent used in step C of the invention can be the same solvent used in step A and step B. A water immiscible solvent can be used to solubilize the intermediate of Formula 4 and the two phase system is agitated by stirring and by boiling of the reaction mixture. When the hydrolysis/decarboxylation is complete the mixture is cooled and the pressure returned to ambient, then the phase containing the compound of Formula 1 can be separated from water in the two phase system. Example 1 demonstrates this method with chlorobenzene. Alternatively the intermediate of Formula 4 can be dissolved in a solvent different from that of step A and that solvent may be a water miscible solvent (e.g. acetonitrile or N, N-dimethylformamide). The hydrolysis/-decarboxylation is then conducted in a one phase system and the compound of Formula 1 can be recovered by concentration of the solvent or extraction with a water immiscible solvent (e.g. diethyl ether or ethyl acetate/hexane mixture). Example 4 demonstrates this method with acetonitrile. Reaction progress can be monitored by conventional methods such as thin layer chromatography, GC, HPLC and ¹H NMR analyses of aliquots. The final solution contains the compound of Formula 1. This solution can be concentrated to isolate the compound of Formula 1 or the compound of Formula 1 in a solvent solution can be carried on to the next synthetic step for which it was intended.

The hydrolysis of the ester groups in the compound of Formula 4 can be accomplished under acidic conditions with water and an acid. The hydrolysis reaction can be run under a broad range of temperatures. Temperatures in the range of 85 to 180° C., are particularly useful. Acid catalyzes the hydrolysis reaction, therefore, reaction can be run at lower temperatures and ambient pressure. Temperatures in the range of 85 to 130° C. are especially useful in order to complete the hydrolysis in a reasonable period of time (several hours). The reaction is conducted in Examples 2 and 3 between 90 to 100° C. and it is complete in 4 to 8 hours. A variety of acids can be used for the hydrolysis/decarboxylation reaction. Useful acids include sulfuric acid, arylsulfonic acids, carboxylic acids and mixtures thereof. Mixtures of acetic acid and sulfuric acid can be used in combination with water and are known in the literature (G. A. Reynolds et. al. Organic Synthesis, 1950, 30, 70-72). Sulfuric acid and water is demonstrated in Example 3 and sulfuric acid/acetic acid and water is demonstrated in Example 2. The acids are catalytic in function and can be use in less than one equivalent amounts but at least 10 mole % is especially useful. Excess acid can help reduce reaction time. When acid is used in the hydrolysis/decarboxylation step, then the acid can be neutralized before separation and isolation of the compound of Formula 1. A useful method involves neutralization of the acid when acetic acid is used because it is soluble in both the organic phase and the aqueous phase. Another useful method involves just separation of the organic and aqueous phases without neutralization when only aqueous sulfuric acid is used. Reaction progress can be monitored by conventional methods such as thin layer chromatography, GC, HPLC and ¹H NMR analyses of aliquots.

Step B of the method of the invention involves the formation of the neutral intermediate of Formula 4 by the acidification of the salt of Formula 4s and is shown in Scheme 2. The compound of Formula 4s (salt of a compound of Formula 4) is the immediate product of Step A of the invention.

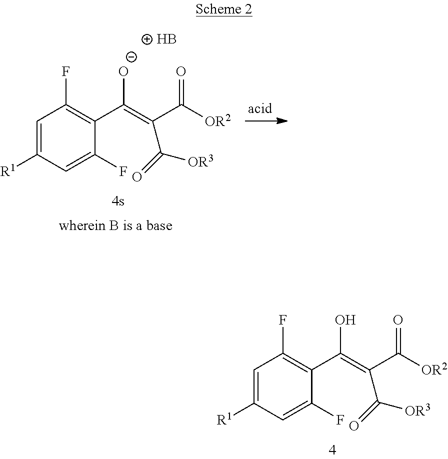

The compound of Formula 4 used in step C of the invention is prepared from the compound of Formula 4s in step B of the invention. The salt resulting from the reaction in step A of the invention is neutralized in step B by contacting the compound of Formula 4s with acid and water to yield the compound of Formula 4. Acids typically used for the neutralization reaction in step B are mineral acids. Acids that are particularly useful are hydrochloric acid and sulfuric acid. The stoichiometry of the neutralization reaction is such that enough acid is added to at least protonate all the equivalents of base added in step A. Most typically a range of 3.0:1.0 to 4.0:1.0 of acid to the compound of Formula 2 (used as the easily measurable reference reagent for stoichiometry). The neutralization reaction is most typically run between 0 and 25° C. A particularly useful method is to cool the reaction mixture from step A to between 0 and 15° C. and add the aqueous acid. Another useful method is to pour the cooled reaction mixture into a separate vessel containing aqueous acid. This method enables a controlled neutralization to give the neutral intermediate compound of Formula 4. The salt of Formula 4s is neutralized in the aprotic solvent that it was prepared in step A of the invention. The aprotic solvent containing the compound of Formula 4, after the neutralization is complete, may be carried on into step C or may be concentrated to isolate the intermediate compound of Formula 4 as an oil. Examples 1 through 3 and 6 through 10 demonstrate use of the same solvent for steps A, B and C (chlorobenzene). Example 4 demonstrates step A and B in the original aprotic solvent and then change of solvent for step C. The intermediate compound of Formula 4 can be isolated and characterized as demonstrated in Example 12.

Step A of the method of the invention involves the reaction of the enolate of the compound of Formula 3 with the acid chloride compound of Formula 2 to give the salt compound of Formula 4s as shown in Scheme 3.

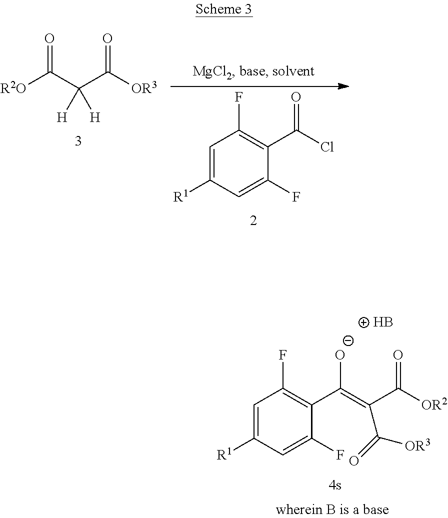

The reagents of step A of the invention can be combined in a variety of orders to prepare the salt of the intermediate of Formula 4 (Formula 4s). A particularly useful method is to prepare the enolate of the compound of Formula 3 first and then add the compound of Formula 2 to it. The preparation of the enolate of the compound of Formula 3 can be accomplished in a variety of orders of addition of the reactants. A particularly useful method is to treat the compound of Formula 3 first with an alkaline earth salt of a strong acid and then add a tertiary amine base. Typically the compound of Formula 3 is dissolved in the aprotic solvent, treated with the alkaline earth salt of a strong acid and a tertiary amine base in sequence and the mixture is allowed to stir for 15 to 60 minutes to form the enolate of the compound of Formula 3. The compound of Formula 2 is then added to the enolate solution and the reaction is allowed to stir for several hours, forming the intermediate of Formula 4. The intermediate of Formula 4 is very acidic and reacts with the base present to form a salt of Formula 4s.

Typically the alkaline earth salt of a strong acid is either magnesium chloride or calcium chloride, most typically magnesium chloride is used. The method used in step A is proposed to generate a magnesium enolate when magnesium chloride is employed (M. W. Rathke et al., Journal of Organic Chemistry 1985, 50, 2622-2624). The alkaline earth salt of a strong acid is critical to enabling the tertiary amine base to completely deprotonate the diester compounds of Formula 3. Calcium chloride can be used alternatively to magnesium chloride (DE 4138616, May 27, 1993). Useful tertiary amine bases for the method of step A include of tributylamine, triethylamine, diisopropylethylamine, pyridine, picolines, lutidines, N,N-dimethylaniline and N,N-diethylaniline. The use of tributylamine, pyridine, 2-picoline, 2,6-lutidine and N,N-diethylaniline are demonstrated in Examples 6 through 10. Triethylamine is especially useful as the tertiary amine base and is demonstrated in Examples 1 through 4.

The reaction of step A is run in the presence of an aprotic solvent. Useful aprotic solvents include chlorobenzene, toluene, xylenes, dichloromethane, tetrahydrofuran, acetonitrile and ethyl acetate. Chlorobenzene and ethyl acetate are especially useful because they are also water immiscible and facilitate the separation of the intermediate of Formula 4 and product of Formula 1 from aqueous phases during steps B and C in the method of the invention. Chlorobenzene also has the advantage of a relatively high boiling point which is a useful property for the hydrolysis step C which involves heating to temperatures in the range of 85 to 180° C. The use of chlorobenzene as the aprotic solvent is demonstrated in Example 1. The use of ethyl acetate as the aprotic solvent is demonstrated in Example 4.

A useful temperature range for step A of the method of the invention is 0 to 25° C. This temperature range is useful for both the reaction of the compound of Formula 3 with the alkaline earth salt of a strong acid and the tertiary amine base and the further reaction of the resultant enolate with the acid chloride of Formula 2. Both formation of the etiolate and reaction of the enolate with the acid chloride can be performed at the low end of the temperature range (0 to 5° C.) or the high end of the temperature range (20 to 25° C.). Another useful mode of reaction is to form the enolate at the high end of the temperature range and react it with the acid chloride at the low end of the temperature range. External cooling may be needed on a large scale to keep the reaction mixture below 25° C.

The stoichiometry of the reaction is measured in reference to the acid chloride of Formula 2. The acid chloride of Formula 2 is often the most expensive reagent and is considered the limiting reagent of step A, whereas the compound of Formula 3 is often cheaper and commercially available. A useful range of ratios of the compound of Formula 3 to the compound of Formula 2 is 1.5:1.0 to 10:1,0. A ratio in the range of 1.5:1.0 to 1.2:1.0 is especially useful because it ensures complete reaction of the compound of Formula 2. A useful ratio of the alkaline earth salt of a strong acid (usually magnesium chloride) to the compound of Formula 2 is 3.5:1.0 to 3.0:1.0. Also, a useful ratio of the tertiary amine base to the compound of Formula 2 is 3.5:1.0 to 3.0:1.0. The excess of a tertiary amine base relative to the malonate of Formula 3 ensures complete formation of the enolate and complete conversion of the compound of Formula 2 to the intermediate of Formula 4. It also provides for the extra equivalent of base to react with the acidic intermediate of Formula 4 to generate the salt of Formula 4s.

The complete formation of the salt of Formula 4s can be determined by acidification of an aliquot of the reaction mixture and analysis by conventional methods such as thin layer chromatography. GC, HPLC and ¹H NMR. The solution containing the salt of Formula 4s can then be treated as in step B of the method of the invention.

In the method of the invention, a compound of Formula 5 and a compound of Formula 2 are reacted to form a monoester intermediate of Formula 6. The monoester intermediate of Formula 6 is hydrolyzed and decarboxylated to give the compound of Formula 1. This sequence is shown in Schemes 4, 5 and 6.

Step C of the method of the invention involves hydrolysis of the ester group in an intermediate of Formula 6 and decarboxylation of the resultant carboxylic acid functional group to give a compound of Formula 1 as shown in Scheme 4.

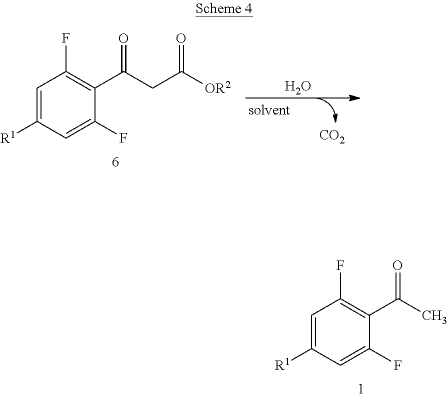

The hydrolysis of the ester group in the compound of Formula 6 can be accomplished under neutral conditions with water. The hydrolysis reaction can be run under a broad range of temperatures. Temperatures in the range of 85 to 180° C. are particularly useful. The lower the temperature used for the hydrolysis, the longer the reaction will take to complete. Therefore temperatures in the range of 130 to 160° C. are especially useful in order to complete the hydrolysis in a reasonable period of time (less than an hour to several hours). The reaction is conducted in Examples 5 and 11 between 135 to 155° C. and it is complete in 1 to 2 hours. When the ester hydrolysis/decarboxylation is conducted with water under neutral conditions at temperatures above the boiling point of water, it is especially useful to run the reaction in a pressure reactor. The pressure reactor can be equipped with a back pressure regulator to enable maintenance of constant pressure while carbon dioxide is evolved and a condenser to return water or solvent to the reaction mixture containing the intermediate of Formula 6.

The hydrolysis reaction requires at least one equivalent of water for every equivalent of a compound of Formula 6, however, an excess of water can be useful to decrease reaction time. The hydrolysis/decarboxylation reaction can be conducted either in a one phase homogeneous solution or a two phase system. The solvent used in step C of the invention can be the same solvent used in step A and step B. A water immiscible solvent can be used to solubilize the intermediate of Formula 6 and the two phase system is agitated by stirring and by boiling of the reaction mixture. When the hydrolysis/decarboxylation is complete the mixture is cooled and the pressure returned to ambient, then the phase containing the compound of Formula 1 can be separated from water in the two phase system. Alternatively the intermediate of Formula 6 can be dissolved in a solvent different from that of step A and that solvent may be a water miscible solvent (e.g. acetonitrile or N, N-dimethylformamide). The hydrolysis/decarboxylation is then conducted in a one phase system and the compound of Formula 1 can be recovered by concentration of the solvent or extraction with a water immiscible solvent (e.g. diethyl ether or ethyl acetate/hexane mixture). Examples 5 and 11 demonstrate this method with acetonitrile and N,N-dimethylformamide respectively. Reaction progress can be monitored by conventional methods such as thin layer chromatography, GC, HPLC and ¹H NMR analyses of aliquots. The final solution contains the compound of Formula 1. This solution can be concentrated to isolate the compound of Formula 1 or the compound of Formula 1 in a solvent solution can be carried on to the next synthetic step for which it was intended.

The hydrolysis of the ester groups in the compound of Formula 6 can be accomplished under acidic conditions with water and an acid. The hydrolysis reaction can be run under a broad range of temperatures. Temperatures in the range of 85 to 180° C. are particularly useful. Acid catalyzes the hydrolysis reaction, therefore, reaction can be run at lower temperatures and ambient pressure. Temperatures in the range of 85 to 130° C. are especially useful in order to complete the hydrolysis in a reasonable period of time (several hours). A variety of acids can be used for the hydrolysis/decarboxylation reaction. Useful acids include sulfuric acid, arylsulfonic acids, carboxylic acids and mixtures thereof. Mixtures of acetic acid and sulfuric acid can be used in combination with water and are known in the literature (G. A. Reynolds et. al. Organic Synthesis, 1950, pages 70-72) The acids are catalytic in function and can be use in less than one equivalent amounts but at least 10 mole % is especially useful. Excess acid can help reduce reaction time. When acid is used in the hydrolysis/decarboxylation step, then the acid can be neutralized before separation and isolation of the compound of Formula 1. Reaction progress can be monitored by conventional methods such as thin layer chromatography, GC, HPLC and ¹H NMR analyses of aliquots.

Step B of the method of the invention involves the formation of the neutral intermediate of Formula 6 by the acidification of the salt of Formula 6s and is shown in Scheme 5. The compound of Formula 6s (salt of a compound of Formula 6) is the immediate product of Step A of the invention.

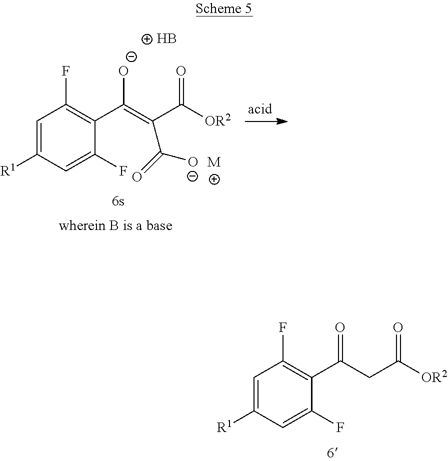

The compound of Formula 6 used in step C of the invention is prepared from the compound of Formula 6s in step B of the invention. The salt resulting from the reaction in step A of the invention is neutralized in step B by contacting the compound of Formula 6s with acid and water to yield the compound of Formula 6. Acids typically used for the neutralization reaction in step B are mineral acids. Acids that are particularly useful are hydrochloric acid and sulfuric acid. The stoichiometry of the neutralization reaction is such that enough acid is added to at least protonate all the equivalents of base added in step A. Most typically a range of 3.0:1.0 to 4.0:1.0 of acid to the compound of Formula 2 (used as the easily measurable reference reagent for stoichiometry). The neutralization reaction is most typically run between 0 and 25° C. A particularly useful method is to cool the reaction mixture from step A to between 0 and 15° C. and add the aqueous acid. Another useful method is to pour the cooled reaction mixture into a separate vessel containing aqueous acid. This method enables a controlled neutralization to give the neutral intermediate compound of Formula 6. The salt of Formula 6s is neutralized in the aprotic solvent that it was prepared in step A of the invention. The aprotic solvent containing the compound of Formula 6, after the neutralization is complete, may be carried on into step C or may be concentrated to isolate the intermediate compound of Formula 6 as an oil. Examples 5 and 11 demonstrate step A and B in the original aprotic solvent and then change of solvent for step C. The intermediate compound of Formula 6 can be isolated and characterized.

Step A of the method of the invention involves the reaction of the enolate of the compound of Formula 5 with the acid chloride compound of Formula 2 to give the salt compound of Formula 6s as shown in Scheme 6.

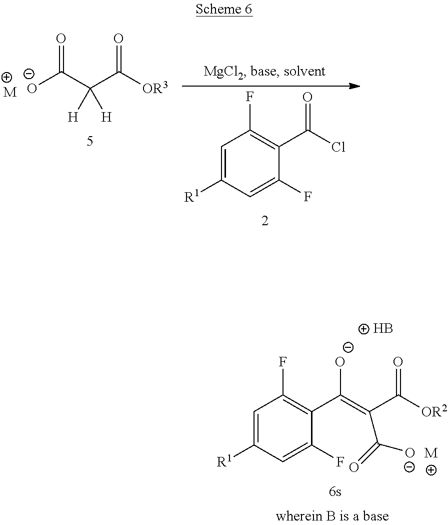

The reagents of step A of the invention can be combined in a variety of orders to prepare the salt of the intermediate of Formula 6 (Formula 6s). A particularly useful method is to prepare the etiolate of the compound of Formula 5 first and then add the compound of Formula 2 to it. The preparation of the enolate of the compound of Formula 5 can be accomplished in a variety of orders of addition of the reactants. A particularly useful method is to treat the compound of Formula 5 first with an alkaline earth salt of a strong acid and then add a tertiary amine base. Typically the compound of Formula 5 is dissolved in the aprotic solvent, treated with the alkaline earth salt of a strong acid and a tertiary amine base in sequence and the mixture is allowed to stir for 15 to 60 minutes to form the enolate of the compound of Formula 5. The compound of Formula 2 is then added to the enolate solution and the reaction is allowed to stir for several hours, forming the intermediate of Formula 6. The intermediate of Formula 6 is acidic and reacts with the base present to form a salt of Formula 6s.

The variable M in the compound of Formula 5 can be lithium, sodium or potassium. It is especially useful to use the potassium counter cation for the compound of Formula 5 because of its higher solubility in organic solvents.

Typically the alkaline earth salt of a strong acid is either magnesium chloride or calcium chloride, most typically magnesium chloride is used. The alkaline earth salt of a strong acid is critical to enabling the tertiary amine base to completely deprotonate the monoester compounds of Formula 5. The use of a tertiary amine base enables the use of milder reaction conditions than other bases known in the art (A. Hashimoto et al., Org. Process Res. Dev. 2007, 11, 389-398). Useful tertiary amine bases for the method of step A include of tributylamine, triethylamine, diisopropylethylamine, pyridine, picolines, lutidines, N,N-dimethylaniline and N,N-diethylaniline. Triethylamine is especially useful as the tertiary amine base and is demonstrated in Examples 5 and 11.

The reaction of step A is run in the presence of an aprotic solvent. Useful aprotic solvents include chlorobenzene, toluene, xylenes, dichloromethane, tetrahydrofuran, acetonitrile and ethyl acetate. Chlorobenzene and ethyl acetate are especially useful because they are also water immiscible and facilitate the separation of the intermediate of Formula 6 and product of Formula 1 from aqueous phases during steps B and C in the method of the invention. Ethyl acetate and tetrahydrofuran also has the advantage of being relatively polar and are better able to solubilize the compound of Formula 5, its dianionic enolate and the dianionic compound of Formula 6s. The use of an ethyl acetate and tetrahydrofuran mixture as the aprotic solvent is demonstrated in Example 5. The use of ethyl acetate as the aprotic solvent is demonstrated in Example 11.

A useful temperature range for step A of the method of the invention is 0 to 50° C. This temperature range is useful for both the reaction of the compound of Formula 5 with the alkaline earth salt of a strong acid and the tertiary amine base and the further reaction of the resultant enolate with the acid chloride of Formula 2. The formation of the enolate is typically performed at the high end of the temperature range (20 to 50° C.) because of difficulty involved in forming a dianionic species. The reaction of the enolate with the acid chloride is typically performed at the low end of the temperature range (0 to 5° C.). External cooling may be needed on a large scale to keep the reaction mixture below 25° C.

The stoichiometry of the reaction is measured in reference to the acid chloride of Formula 2. The acid chloride of Formula 2 is often the most expensive reagent and is considered the limiting reagent of step A, whereas the compound of Formula 5 is often cheaper and commercially available. A useful range of ratios of the compound of Formula 5 to the compound of Formula 2 is 1.5:1.0 to 1.0:1.0. A ratio in the range of 1.5:1.0 to 1.2:1.0 is especially useful because it ensures complete reaction of the compound of Formula 2. A useful ratio of the alkaline earth salt of a strong acid (usually magnesium chloride) to the compound of Formula 2 is 3.5:1.0 to 3.0:1.0. Also, a useful ratio of the tertiary amine base to the compound of Formula 2 is 3.5:1.0 to 3.0:1.0. The excess of a tertiary amine base relative to the ester/carboxylate of Formula 5 ensures complete formation of the enolate and complete conversion of the compound of Formula 2 to the intermediate of Formula 6. It also provides for the extra equivalent of base to react with the acidic intermediate of Formula 6 to generate the salt of Formula 6s.

The complete formation of the salt of Formula 6s can be determined by acidification of an aliquot of the reaction mixture and analysis by conventional methods such as thin layer chromatography, GC, HPLC and ¹H NMR. The solution containing the salt of Formula 6s can then be treated as in step B of the method of the invention.

Without further elaboration, it is believed that one skilled in the art using the preceding description can utilize the present invention to its fullest extent. The following Examples are, therefore, to be construed as merely illustrative, and not limiting of the disclosure in any way whatsoever. Steps in the following Examples illustrate a procedure for each step in an overall synthetic transformation, and the starting material for each step may not have necessarily been prepared by a particular preparative run whose procedure is described in other Examples or Steps.

HPLC analyses were performed using a Hewlett Packard 1100 series HPLC system with DAD/UV detector and reverse-phase column (Agilent Eclipse XDB-C8 (4.6×150) mm, 5 μm, Part. No. 993967-906). Flow rate was 1.0 mL/min, run time 25 min, injection volume 3.0 μL, and the column temperature was 40° C. Mobile phase A was 0.075% orthophosphoric acid (aq) and mobile phase B was acetonitrile (HPLC grade). For wt % determination the concentration of the test sample was calibrated against a standard sample.

¹H NMR spectra are reported in ppm downfield from tetramethylsilane and ¹⁹F NMR spectra are reported in ppm upfield from CFCl₃; “s” means singlet, “d” means doublet, “t” means triplet, “q” means quartet, “in” means multiplet, “dd” means doublet of doublets, “dt” means doublet of triplets and “br” means broad.

### Example 1

### Preparation of 2,6-Difluoroacetophenone

Magnesium chloride (167 g, 1.75 mal) was added to a solution of diethyl malonate (125 g, 780 mmol) in chlorobenzene (500 mL) and the slurry was stirred at ambient temperature for 30 minutes. Triethylamine (238 mL, 1.71 mol) was added with external cooling keeping the internal temperature between 25-27° C. during the addition. The slurry was stirred for 30 min at ambient temperature. A solution of 2,6-difluorobenzoyl chloride (100 g, 565 mmol) in chlorobenzene (100 mL) was added slowly with external cooling keeping the temperature between 25-27° C. during the addition. The slurry was stirred for 2 hours at ambient temperature and then cooled to 0° C. The slurry was poured into 1N hydrochloric acid (2000 mL). The biphasic mixture was allowed to return to ambient temperature and the phases allowed to separate. The chlorobenzene (bottom) phase was removed and transferred to a pressure reactor with a condenser and backpressure regulator. Water (200 mL) was added to the mixture and the reaction was sealed. The reaction was stirred and heated to 140° C. for 2 hours. The reaction was cooled to ambient temperature and the residual pressure was released. The phases were allowed to separate and the chlorobenzene (bottom) phase containing the title compound was separated. HPLC wt % analysis of this solution indicated a 2,6-difluoroacetophenone yield of 84.6 g (96%).

### Example 2

### Preparation of 2,6-Difluoroacetophenone: Hydrolysis with Sulfuric Acid/Acetic Acid

Magnesium chloride (167 g, 1.75 mol) was added to a solution of diethyl malonate (12.5 g, 780 mmol) in chlorobenzene (500 mL) and the slurry was stirred at ambient temperature for 30 minutes Triethylamine (238 mL, 1.71 mol) was added with external cooling keeping the internal temperature between 25-27° C. during the addition. The slurry was stirred for 30 minutes at ambient temperature. A solution of 2,6-difluorobenzoyl chloride (100 g, 565 mmol) in chlorobenzene (100 mL) was added slowly with external cooling keeping the temperature between 25-27° C. during the addition. The slurry was stirred for 2 hours at ambient temperature then cooled to 0° C. The slurry was poured into 1N hydrochloric acid (2000 mL). The biphasic mixture was allowed to return to ambient temperature and the phases allowed to separate. The phases were separated. To a portion of the chlorobenzene phase (76 g) was added a mixture of concentrated sulfuric acid (10 mL) and 60% aqueous acetic acid (35 mL). The mixture was heated to 91-94° C. for 7 hours, cooled to ambient temperature and then adjusted to pH 7 with 10% aq. sodium hydroxide. The phases were separated and the aqueous phase was back extracted with chlorobenzene. The chlorobenzene phases were combined and washed with water. HPLC wt. % analysis of the combined chlorobenzene phases indicated a 2,6-ditluoroacetophenone yield of 7.57 g (87%).

### Example 3

### Preparation of 2,6-Difluoroacetophenone: Hydrolysis with Sulfuric Acid

Magnesium chloride (167 g, 1.75 mol) was added to a solution of diethyl malonate (125 g, 780 mmol) in chlorobenzene (500 mL) and the slurry was stirred at ambient temperature for 30 minutes. Triethylamine (238 mL, 1.71 mol) was added with external cooling keeping the internal temperature between 25-27° C. during the addition. The slurry was stirred for 30 minutes at ambient temperature. A solution of 2,6-difluorobenzoyl chloride (100 g, 565 mmol) in chlorobenzene (100 mL) was added slowly with external cooling keeping the temperature between 25-27° C. during the addition. The slurry was stirred for 2 hours at ambient temperature then cooled to 0° C. The slurry was poured into 1N hydrochloric acid (2000 mL). The biphasic mixture was allowed to return to ambient temperature and the phases allowed to separate. The phases were separated. To a portion of the chlorobenzene phase (76 g) was added 75% aqueous sulfuric acid (40 g). The mixture was stirred and heated to 91-94° C. for 4 hours. The mixture was cooled to ambient temperature and the phases allowed to separate. The chlorobenzene phase was removed. HPLC wt % analysis of the chlorobenzene phase indicated a 2,6-difluoroacetophenone yield of 7.36 g (85%).

### Example 4

### Preparation of 2,6-difluoroacetophenone: Hydrolysis with Acetonitrile/Water

Magnesium chloride (1.65 g, 17.3 mmol) was added to a solution of diethyl malonate (1.24 g, 7.7 mmol) in ethyl acetate (20 mL) and the slurry was stirred at ambient temperature for 30 minutes. Triethylamine (2.35 mL, 16.7 mmol) was added and the slurry was stirred for another 30 minutes. The slurry was cooled to 0° C. and a solution of 2,6-difluorobenzoyl chloride (1.0 g, 5.6 mmol) in ethyl acetate (5 mL) was added dropwise over 15 minutes maintaining the internal temperature below 5° C. At the end of the addition the reaction was allowed to warm to ambient temperature and stirred for approximately 3 hours. The slurry was then treated with 1N hydrochloric acid (50 mL) and extracted with ethyl acetate (100 mL). The organic phase was separated, dried over MgSO₄, and filtered. The filtrate was concentrated under reduced pressure yielding a colorless oil (1.97 g) containing the intermediate. The oil was dissolved in acetonitrile (25 mL) and water (2 mL) was added. The solution was transferred to a pressure reactor and sealed. The intermediate solution was stirred and heated to 150° C. for 1 hour. The reaction mixture was cooled to ambient temperature and the residual pressure released. HPLC wt % analysis of the solution indicated a 2,6-difluoroacetophenone yield of 874 mg (100%).

### Example 5

### Preparation of 2,6-Difluoroacetophenone Using Ethyl Malonate, Potassium Salt

Ethyl malonate, potassium salt (13.4 g, 77 mmol), magnesium chloride (16.5 g, 173 mmol), ethyl acetate (40 mL) and tetrahydrofuran (60 mL) were combined and stirred for 30 minutes at ambient temperature. The reaction mixture was cooled to 0° C. and triethylamine (23.5 mL, 167 mmol) was added. The reaction slurry was heated to 50° C. and held for 1 hour, then cooled back to 0° C. A solution of 2,6-difluorobenzoyl chloride (10.0 g, 56 mmol) in ethyl acetate (25 mL) was added slowly to the slurry over 55 min maintaining the internal temperature below 2° C. At the end of the addition the reaction was allowed to warm to ambient temperature and stirred for 19 hours. The reaction was cooled to 0° C. and treated with 1N hydrochloric acid (200 mL). The clear biphasic mixture was allowed to return to ambient temperature and additional ethyl acetate (100 mL) was added. The phases allowed to separate and the organic phase was dried over MgSO₄, filtered and the filtrate concentrated under reduced pressure, yielding a yellow oil residue (15.46 g) containing the intermediate. The oil was dissolved in acetonitrile (100 mL) and water (5 mL) and transferred to a pressure reactor with a condenser and backpressure regulator. The reaction mixture was sealed in the pressure reactor, stirred and heated to 1.50° C. for 1 hour. The reaction was cooled to ambient temperature and the residual pressure was released. HPLC wt % analysis of the reaction solution indicated a 2,6-difluoroacetophenone yield of 8.60 g (99%).

### Example 6

### Preparation of 2,6-Difluoroacetophenone Using Pyridine as the Base

Magnesium chloride (1.65 g, 17.3 mmol) was added to a solution of diethyl malonate (1.24 g, 7.7 mmol) in chlorobenzene (20 mL) and the slurry was stirred at ambient temperature for 31) minutes. Pyridine (1.35 mL, 16.7 mmol) was added and the slurry was stirred for another 30 minutes. The reaction was cooled to 0° C. and a solution of 2,6-difluorobenzoyl chloride (1.0 g, 5.6 mmol) in chlorobenzene (5 mL) was added dropwise over approximately 10 min keeping the internal temperature below 1° C. The reaction was allowed to warm to ambient temperature and stirred for approximately 21 hours. The reaction mixture was treated with 1N hydrochloric acid (20 mL) and diluted with water (80 mL). The phases were allowed to separate and the chlorobenzene (bottom) phase was transferred to a pressure reactor. Water (2 mL) was added to the reactor and the reactor was sealed. The reaction mixture was stirred and heated to 150° C. for 1 hour. The reaction was cooled to ambient temperature and the residual pressure was released. The reaction mixture was diluted with additional water and chlorobenzene and the phases allowed to separate. The chlorobenzene (bottom) phase containing the title compound was separated. HPLC wt % analysis of the chlorobenzene phase indicated a 2,6-difluoroacetophenone yield of 505 mg (58%).

### Example 7

### Preparation of 2,6-Difluoroacetophenone Using 2,6-Lutidine as the Base

Magnesium chloride (1.65 g, 17.3 mmol) was added to a solution of diethyl malonate (1.24 g, 7.7 mmol) in chlorobenzene (20 mL) and the slurry was stirred at ambient temperature for 30 minutes. 2,6-Lutidine (1.93 mL, 16.7 mmol) was added and the slurry was stirred for another 30 minutes. The reaction was cooled to 0° C. and a solution of 2,6-difluorobenzoyl chloride (1.0 g, 5.6 mmol) in chlorobenzene (5 mL) was added dropwise over approximately 10 minutes keeping the internal temperature below 1° C. The reaction was allowed to warm to ambient temperature and stir for approximately 21 hours. The reaction was treated with 1N hydrochloric acid (50 mL) and diluted with water (50 mL). The phases allowed to separate and the chlorobenzene (bottom) phase was transferred to a pressure reactor. Water (2 mL) was added to the reactor and the reactor was sealed. The reaction mixture was stirred and heated to 150° C. for 1 hour. The reaction was cooled to ambient temperature and the residual pressure was released. The reaction mixture was diluted with additional water and chlorobenzene and the phases allowed to separate. The chlorobenzene (bottom) phase containing the title compound was separated. HPLC wt % analysis of the chlorobenzene phase indicated, a 2,6-difluoroacetophenone yield of 859 mg (99%).

### Example 8

### Preparation of 2,6-Difluoroacetophenone Using 2-Picoline as the Base

Magnesium chloride (1.65 g, 17.3 mmol) was added to a solution of diethyl malonate (1.24 g, 7.7 mmol) in chlorobenzene (20 mL) and the slurry was stirred at ambient temperature for 30 minutes. 2-Picoline (1.68 mL, 16.7 mmol) was added and the slurry was stirred for another 30 minutes. The reaction was cooled to 0° C. and a solution of 2,6-difluorobenzoyl chloride (1.0 g, 5.6 mmol) in chlorobenzene (5 mL) was added dropwise to the reaction over approximately 10 min keeping the internal temperature below 1° C. The reaction was allowed to warm to ambient temperature and stirred for approximately 24 hours. The reaction was treated with 1N hydrochloric acid (50 mL) and diluted with water (50 mL). The phases allowed to separate and the chlorobenzene (bottom) phase was transferred to a pressure reactor. Water (2 mL) was added to the reactor and the reactor was sealed. The reaction mixture was stirred and heated to 150° C. for 1 hour. The reaction was cooled to ambient temperature and the residual pressure was released. The reaction mixture was diluted with additional water and chlorobenzene and the phases were allowed to separate. The chlorobenzene (bottom) layer containing the title compound was separated. HPLC wt % analysis of the chlorobenzene phase indicated a 2,6-difluoroacetophenone yield of 697 mg (80%).

### Example 9

### Preparation of 2,6-Difluoroacetophenone Using N,N-Diethylaniline as the Base

Magnesium chloride (1.65 g, 17.3 mmol) was added to a solution of diethyl malonate (1.24 g, 7.7 mmol) in chlorobenzene (20 mL) and the slurry was stirred at ambient temperature for 30 minutes. N,N-Diethylaniline (2.65 mL, 16.7 mmol) was added and the slurry was stirred for another 30 minutes. The reaction was cooled to 0° C. and a solution of 2,6-difluorobenzoyl chloride (1.0 g, 5.6 mmol) in chlorobenzene (5 mL) was added dropwise over 10 minutes keeping the internal temperature below 1° C. The reaction mixture was allowed to warm to ambient temperature and stirred for 22 hours. The reaction was treated with 1N hydrochloric acid (50 mL) and diluted with water (50 mL). The phases were allowed to separate and the chlorobenzene (bottom) phase was transferred to a pressure reactor. Water (2 mL) was added to the reactor and the reactor was sealed. The reaction mixture was stirred and heated to 150° C. for 1 hour. The reaction was cooled to ambient temperature and the residual pressure was released. The reaction mixture was diluted with additional water and chlorobenzene and the phases were allowed to separate. The chlorobenzene (bottom) phase containing the title compound was separated. HPLC wt % analysis of the chlorobenzene phase indicated, a 2,6-difluoroacetophenone yield of 876 mg (100%).

### Example 10

### Preparation of 2,6-Difluoroacetophenone Using Tributylamine as the Base

Magnesium chloride (1.65 g, 17.3 mmol) was added to a solution of diethyl malonate (1.24 g, 7.7 mmol) in chlorobenzene (20 mL) and the slurry was Mined at ambient temperature for 30 minutes. Tributylamine (1.98 mL, 16.7 mmol) was added and the slurry was stirred for another 30 minutes. The reaction was cooled to 0° C. and a solution of 2,6-difluorobenzoyl chloride (1.0 g, 5.6 mmol) in chlorobenzene (5 mL) was added dropwise to the reaction over approximately 10 minutes keeping the internal temperature below 1° C. The reaction was allowed to warm to ambient temperature and stirred for 22 hours. The reaction mixture was treated with 1N hydrochloric acid (50 mL) and diluted with water (50 mL). The phases were allowed to separate and the chlorobenzene (bottom) phase was transferred to a pressure reactor. Water (2 mL) was added to the reactor and the reactor was sealed. The reaction was stirred and heated to 150° C. for 1 hour. The reaction was cooled to ambient temperature and the residual pressure was released. The reaction mixture was diluted with additional water and chlorobenzene and the phases were allowed to separate. The chlorobenzene (bottom) phase containing the title compound was separated. HPLC wt % analysis of the chlorobenzene phase indicated a 2,6-difluoroacetophenone yield of 701 mg (81%).

### Example 11

### A Second Preparation of 2,6-Difluoroacetophenone Using Ethyl Malonate, Potassium Salt

Magnesium chloride (16.5 g, 173 mmol) was added to a slurry of ethyl malonate, potassium salt (13.1 g, 77 mmol) in ethyl acetate (80 mL) and the slurry was stirred for 30 mm at ambient temperature and then cooled to 0° C. Triethylamine (23.5 mL, 167 mmol) was added and the slurry was heated to 50° C. and held for 2 hours. The slurry was cooled to 0° C. and a solution of 2,6-difluorobenzoyl chloride (10.0 g, 56 mmol) in ethyl acetate (25 mL) was added dropwise over 30 minutes keeping the internal temperature below 5° C. The reaction was allowed to warm to ambient temperature and stir for 18 hours. The reaction mixture was treated with 1N hydrochloric acid (200 mL) and extracted with ethyl acetate (100 mL). The organic phase was separated, dried over MgSO₄ and filtered. The filtrate was concentrated under reduced pressure yielding a pale yellow oil containing the intermediate (15.25 g). The oil was dissolved in NA-dimethylformamide (100 mL) and water (5 mL) was added. The solution was stirred and heated to reflux (135° C.) for approximately 2 hours, then cooled to ambient temperature. The reaction was diluted with water (200 mL) and extracted twice with 250 mL portions of a 5:1 hexanes:ethyl acetate mixture. The organic phases were combined, dried, over MgSO₄ and filtered. The filtrate was concentrated yielding a yellow oil (9.11 g) containing the title compound and residual N,N-dimethylformamide. The oil was dissolved in ethyl acetate (100 mL) and washed twice with 100 mL portions of 1N hydrochloric acid. The organic phase was dried over MgSO₄ and filtered. The filtrate was concentrated yielding a yellow oil (6.54 g, 75% yield) ¹H NMR (CDCl₃) δ 7.45-7.35 (m, 1H), δ 7.00-6.91 (m, 2H), δ 2.61 (t, J=1.8 Hz, 3H). ¹⁹F NMR (CDCl₃) δ −112.02 ppm (m).

### Example 12

### Preparation and isolation of 1,3-diethyl 2(2,6-difluorobenzoyl)propanedioate (keto) and 1,3-diethyl 2-[(2,6-difluorophenyl)hydroxymethylene]propanedioate (enol) (a Compound of Formula 4)

Magnesium chloride (6.7 g, 70 mmol) was added to a solution of diethyl malonate (5 g, 30 mmol) in chlorobenzene (20 mL) and the slurry was stirred at ambient temperature for 30 minutes. Triethylamine (9.5 nit) was added with external cooling keeping the internal temperature between 25-27° C. during the addition. The slurry was stirred for another 30 minutes and then cooled to 0° C. A solution of 2,6-difluorobenzoyl chloride (4 g, 22 mmol) in chlorobenzene (4 mL) was added dropwise keeping the internal temperature between 0-3° C. during the addition. At the end of the addition the reaction was allowed to warm to ambient temperature and stir for 2 hours. The reaction mixture was cooled back to 0° C. and poured into 1N hydrochloric acid (80 mL). The biphasic mixture was allowed to return to ambient temperature and the phases were allowed to separate. The chlorobenzene (bottom) phase was separated. The intermediate was isolated from the chlorobenzene phase by prep HPLC with a purity of 91.56% by GC (A %) and 98.32% by HPLC (A %) as a mixture of tautomers in approximately 5:1 enol:keto form.

¹H NMR (CDCl₃) (mixture) δ 7.53-7.35 (m, 1H), δ 7.02-6.91 (m, 2H);

(keto) δ 5.12 (s, 1H), δ 4.28 (q, J=7.2 Hz, 4H), δ 1.28 (t, J=7.2 Hz, 6H);

¹⁹F NMR (CDCl₃) δ −110.57 ppm (m).

(enol) δ 13.85 (s, 1H), δ 4.38 (q, J=7.3 Hz, 2H), δ 4.02 q, J=7.3 Hz, 2H), δ 1.38 (t, J=7.3 Hz, 3H), δ 0.97 (t, J=7.3 Hz, 3H);

¹⁹F NMR (CDCl₃) δ −111.97 ppm (m).

Table 1 illustrates the particular transformations to prepare compounds of Formula 1 according to a method of the present invention.

TABLE 1

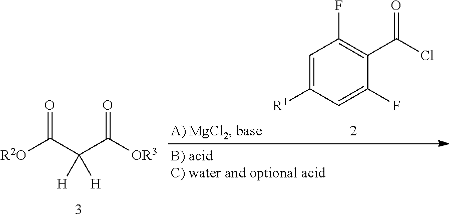

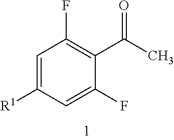

| R¹ | R² | R³ |
|---|---|---|
| H | CH₃ | CH₃ |
| H | CH₃ | CH₂CH₃ |
| H | CH₃ | CH₂CH═CH₂ |
| H | CH₂CH₃ | CH₃ |
| H | CH₂CH₃ | CH₂CH₃ |
| H | CH₂CH₃ | CH₂CH═CH₂ |
| H | CH₂CH═CH₂ | CH₃ |
| H | CH₂CH═CH₂ | CH₂CH₃ |
| H | CH₂CH═CH₂ | CH₂CH═CH₂ |
| H | —C(CH₃)₂— | |
| F | CH₃ | CH₃ |
| F | CH₃ | CH₂CH₃ |
| F | CH₃ | CH₂CH═CH₂ |
| F | CH₂CH₃ | CH₃ |
| F | CH₂CH₃ | CH₂CH₃ |
| F | CH₂CH₃ | CH₂CH═CH₂ |
| F | CH₂CH═CH₂ | CH₃ |
| F | CH₂CH═CH₂ | CH₂CH₃ |
| F | CH₂CH═CH₂ | CH₂CH═CH₂ |
| F | —C(CH₃)₂— | |
| Cl | CH₃ | CH₃ |
| Cl | CH₃ | CH₂CH₃ |
| Cl | CH₃ | CH₂CH═CH₂ |
| Cl | CH₂CH₃ | CH₃ |
| Cl | CH₂CH₃ | CH₂CH₃ |
| Cl | CH₂CH₃ | CH₂CH═CH₂ |
| Cl | CH₂CH═CH₂ | CH₃ |
| Cl | CH₂CH═CH₂ | CH₂CH₃ |
| Cl | CH₂CH═CH₂ | CH₂CH═CH₂ |
| Cl | —C(CH₃)₂— | |
| Br | CH₃ | CH₃ |
| Br | CH₃ | CH₂CH₃ |
| Br | CH₃ | CH₂CH═CH₂ |
| Br | CH₂CH₃ | CH₃ |
| Br | CH₂CH₃ | CH₂CH₃ |
| Br | CH₂CH₃ | CH₂CH═CH₂ |
| Br | CH₂CH═CH₂ | CH₃ |
| Br | CH₂CH═CH₂ | CH₂CH₃ |
| Br | CH₂CH═CH₂ | CH₂CH═CH₂ |

| | |
|---|---|
| Br | —C(CH₃)₂— |

Table 2 illustrates the particular transformations to prepare compounds of Formula 1 according to a method of the present invention.

TABLE 2

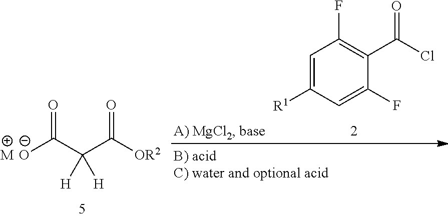

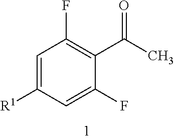

| R¹ | R² | M |
|---|---|---|
| H | CH₃ | Li |
| H | CH₃ | Na |
| H | CH₃ | K |
| H | CH₂CH₃ | Li |
| H | CH₂CH₃ | Na |
| H | CH₂CH₃ | K |
| H | CH₂CH═CH₂ | Li |
| H | CH₂CH═CH₂ | Na |
| H | CH₂CH═CH₂ | K |
| F | CH₃ | Li |
| F | CH₃ | Na |
| F | CH₃ | K |
| F | CH₂CH₃ | Li |
| F | CH₂CH₃ | Na |
| F | CH₂CH₃ | K |
| F | CH₂CH═CH₂ | Li |
| F | CH₂CH═CH₂ | Na |
| F | CH₂CH═CH₂ | K |
| Cl | CH₃ | Li |
| Cl | CH₃ | Na |
| Cl | CH₃ | K |
| Cl | CH₂CH₃ | Li |
| Cl | CH₂CH₃ | Na |
| Cl | CH₂CH₃ | K |
| Cl | CH₂CH═CH₂ | Li |
| Cl | CH₂CH═CH₂ | Na |
| Cl | CH₂CH═CH₂ | K |
| Br | CH₃ | Li |
| Br | CH₃ | Na |
| Br | CH₃ | K |
| Br | CH₂CH₃ | Li |
| Br | CH₂CH₃ | Na |
| Br | CH₂CH₃ | K |
| Br | CH₂CH═CH₂ | Li |
| Br | CH₂CH═CH₂ | Na |
| Br | CH₂CH═CH₂ | K |

Table 3 illustrates the particular intermediate compounds of Formula 4 formed in a method of the present invention. As stated previously, there are several tautomeric forms of the compounds of Formula 4 and illustration of one tautomeric form is meant to represent all tautomeric forms available to the compounds of Formula 4.

TABLE 3

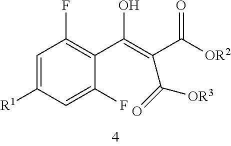

| Cmpd. No. | R¹ | R² | R³ |
|---|---|---|---|
| 4-1 | H | CH₃ | CH₃ |
| 4-2 | H | CH₃ | CH₂CH₃ |
| 4-3 | H | CH₃ | CH₂CH═CH₂ |
| 4-4 | H | CH₂CH₃ | CH₃ |
| 4-5 | H | CH₂CH₃ | CH₂CH₃ |
| 4-6 | H | CH₂CH₃ | CH₂CH═CH₂ |
| 4-7 | H | CH₂CH═CH₂ | CH₃ |
| 4-8 | H | CH₂CH═CH₂ | CH₂CH₃ |
| 4-9 | H | CH₂CH═CH₂ | CH₂CH═CH₂ |
| 4-10 | H | —C(CH₃)₂— | |
| 4-11 | F | CH₃ | CH₃ |
| 4-12 | F | CH₃ | CH₂CH₃ |
| 4-13 | F | CH₃ | CH₂CH═CH₂ |
| 4-14 | F | CH₂CH₃ | CH₃ |
| 4-15 | F | CH₂CH₃ | CH₂CH₃ |
| 4-16 | F | CH₂CH₃ | CH₂CH═CH₂ |
| 4-17 | F | CH₂CH═CH₂ | CH₃ |
| 4-18 | F | CH₂CH═CH₂ | CH₂CH₃ |
| 4-19 | F | CH₂CH═CH₂ | CH₂CH═CH₂ |
| 4-20 | F | —C(CH₃)₂— | |
| 4-21 | Cl | CH₃ | CH₃ |
| 4-22 | Cl | CH₃ | CH₂CH₃ |
| 4-23 | Cl | CH₃ | CH₂CH═CH₂ |
| 4-24 | Cl | CH₂CH₃ | CH₃ |
| 4-25 | Cl | CH₂CH₃ | CH₂CH₃ |
| 4-26 | Cl | CH₂CH₃ | CH₂CH═CH₂ |
| 4-27 | Cl | CH₂CH═CH₂ | CH₃ |
| 4-28 | Cl | CH₂CH═CH₂ | CH₂CH₃ |
| 4-29 | Cl | CH₂CH═CH₂ | CH₂CH═CH₂ |
| 4-30 | Cl | —C(CH₃)₂— | |
| 4-31 | Br | CH₃ | CH₃ |
| 4-32 | Br | CH₃ | CH₂CH₃ |
| 4-33 | Br | CH₃ | CH₂CH═CH₂ |
| 4-34 | Br | CH₂CH₃ | CH₃ |
| 4-35 | Br | CH₂CH₃ | CH₂CH₃ |
| 4-36 | Br | CH₂CH₃ | CH₂CH═CH₂ |
| 4-37 | Br | CH₂CH═CH₂ | CH₃ |
| 4-38 | Br | CH₂CH═CH₂ | CH₂CH₃ |
| 4-39 | Br | CH₂CH═CH₂ | CH₂CH═CH₂ |

| | | |
|---|---|---|
| 4-40 | Br | —C(CH₃)₂— |

1. A method for preparing a compound of Formula 1 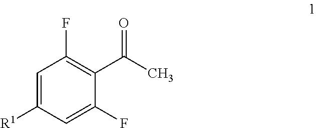
wherein
R¹ is H, F, Cl or Br;
comprising (A) contacting a compound of Formula 2 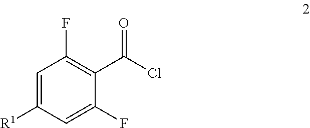
with a compound of Formula 3 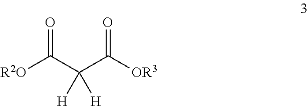
wherein
R² and R³ are independently CH₃, CH₂CH₃, CH₂CH═CH₂ or R² and R³ groups can be taken together as —C(CH₃)₂— to form a ring
and an alkaline earth salt of a strong acid in the presence of a tertiary amine base and an aprotic solvent to form a salt of a compound of Formula 4 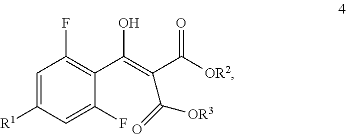
(B) contacting the salt of the compound of Formula 4 with water and an acid to form the compound of Formula 4 or tautomer thereof, and
(C) contacting the compound of Formula 4 with water and heating to a temperature in the range of 85 to 180° C. to give the compound of Formula 1.

2. The method of claim 1 wherein R¹ is H; and R² and R³ are CH₂CH₃.

3. The method of claim 1 wherein the alkaline earth salt of a strong acid is magnesium chloride.

4. The method of claim 1 wherein the tertiary amine base is selected from the group consisting of tributylamine, triethylamine, diisopropylethylamine, pyridine, picolines, lutidines, N,N-dimethylaniline and N,N-diethylaniline.

5. (canceled)

6. The method of claim 1 wherein the aprotic solvent is chlorobenzene, toluene, xylenes, dichloromethane, tetrahydrofuran, acetonitrile or ethyl acetate.

7. (canceled)

8. The method of claim 1 wherein in step (C) the compound of Formula 4 is contacted with water in a pressure reactor and the temperature is in the range of 130 to 160° C.

9. The method of claim 1 wherein in step (C) the compound of Formula 4 is contacted with water in the presence of an acid and heated to a temperature in the range of 85 to 130° C. to give the compound of Formula 1.

10. The method of claim 9 wherein in step (C) the acid is sulfuric acid, acetic acid or mixtures thereof.

11. A compound of Formula 4 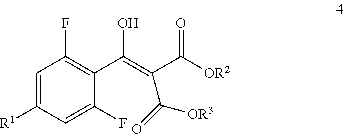
wherein
R¹ is H, F, Cl or Br; and
R² and R³ are independently CH₃, CH₂CH₃, CH₂CH═CH₂ or R² and R³ groups can be taken together as —C(CH₃)₂— to form a ring.

12. (canceled)

13. A method for preparing a compound of Formula 1 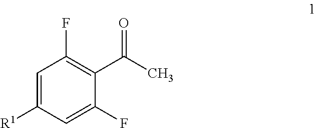
wherein
R¹ is H, F, Cl or Br;
comprising (A) contacting a compound of Formula 2 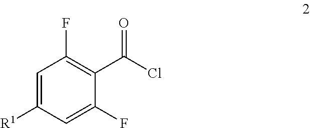
with a compound of Formula 5 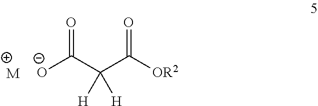
wherein
R² is CH₃, CH₂CH₃ or CH₂CH═CH₂ and
M is Li, Na or K
and an alkaline earth salt of a strong acid in the presence of a tertiary amine base and an aprotic solvent to form a salt of a compound of Formula 6 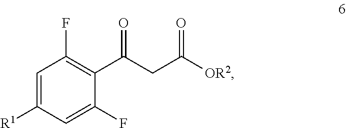
(B) contacting the salt of the compound of Formula 6 with an acid and water to form the compound of Formula 6 or tautomer thereof,
and (C) contacting the compound of Formula 6 with water and heating to a temperature in the range of 85 to 180° C. to give the compound of Formula 1.

14. The method of claim 13 wherein R¹ is H, R² is CH₂CH₃ and M is K.

15. The method of claim 13 wherein the alkaline earth salt of a strong acid is magnesium chloride.

16. The method of claim 13 wherein the tertiary amine base is selected from the group consisting of tributylamine, triethylamine, diisopropylethylamine, pyridine, picolines, lutidines, N,N-dimethylaniline and N,N-diethylaniline.

17. (canceled)

18. The method of claim 13 wherein the aprotic solvent is chlorobenzene, toluene, xylenes, dichloromethane, tetrahydrofuran, acetonitrile or ethyl acetate.

19. (canceled)

20. The method of claim 13 wherein in step (C) the compound of Formula 6 is contacted with water in a pressure reactor and the temperature is in the range of 130 to 160° C.

21. The method of claim 13 wherein in step (C) the compound of Formula 6 is contacted with water in the presence of an acid and heated to a temperature in the range of 85 to 130° C. to give the compound of Formula 1.

22. The method of claim 21 wherein in step (C) the acid is sulfuric acid, acetic acid or mixtures thereof.
